(19) United States
(12) Patent Application Publication
(10) Pub. No.: US 2023/0218345 A1
(43) Pub. Date: Jul. 13, 2023

(54) MEDICAL INFORMATION PROCESSING APPARATUS AND METHOD

(71) Applicant: Canon Medical Systems Corporation, Otawara-shi (JP)

(72) Inventors: Yusuke KANO, Nasushiobara (JP); Anri YAMAZAKI, Nasushiobara (JP)

(73) Assignee: Canon Medical Systems Corporation, Otawara-shi (JP)

(21) Appl. No.: 18/153,434

(22) Filed: Jan. 12, 2023

(30) Foreign Application Priority Data
Jan. 13, 2022 (JP) 2022-003873

(51) Int. Cl. A61B 34/10

(52) U.S. Cl. CPC A61B 34/10

(57) ABSTRACT

According to one embodiment, a medical information processing apparatus includes processing circuitry. The processing circuitry predicts a medical treatment effect of each of a plurality of options that are possibly selected as a medical treatment judgment for a medical care target. The processing circuitry computes, based on a prediction result of the medical treatment effect, a first importance degree relating to an effect common to the options and a second importance degree relating to a difference in effect between the options, with respect to each of one or more features that affect the medical treatment effect. The processing circuitry presents the first importance degree and the second importance degree by one graph or one list.

### CROSS-REFERENCE TO RELATED APPLICATIONS

This application is based upon and claims the benefit of priority from Japanese Patent Application No. 2022-003873, filed Jan. 13, 2022, the entire contents of which are incorporated herein by reference.

### FIELD

Embodiments described herein relate generally to a medical information processing apparatus and method.

### BACKGROUND

There is known a clinical decision support (CDS) in which causal inference is applied. The CDS, in which causal inference is applied, can infer an optimal medical treatment for each individual person from among a plurality of medical treatment options, leading to realization of personalized medicine. In the CDS, importance is placed on explainability or interpretability, and, by the presentation of a recommended medical treatment option and also the basis for the recommended medical treatment option, a patient and a doctor can more easily make judgments with consent.

However, in a case where the importance degree in regard to each medical treatment option is displayed, since the amount of information is too large, there arises a problem that the interpretation of information is difficult. In addition, although there are needs to specify a factor contributing to a difference in effect between medical treatment options, and a factor contributing to an outcome, such as prognosis, regardless of medical treatment options, there is a problem that it is difficult for the doctor to select necessary information for judgment from an enormous amount of information.

### BRIEF DESCRIPTION OF THE DRAWINGS

FIG. 1 is a block diagram illustrating a medical information processing apparatus according to a first embodiment.

FIG. 2 is a flowchart illustrating an operation of the medical information processing apparatus according to the first embodiment.

FIG. 3 is a view illustrating a first presentation example of a first importance degree and a second importance degree.

FIG. 4 is a view illustrating a second presentation example of the first importance degree and the second importance degree.

FIG. 5 is a view illustrating a third presentation example of the first importance degree and the second importance degree.

FIG. 6 is a view illustrating a fourth presentation example of the first importance degree and the second importance degree.

FIG. 7 is a flowchart illustrating details of a process by a computation function in a case where three or more options are present.

FIG. 8 is a view illustrating a second display example in the case where three or more options are present.

FIG. 9 is a view illustrating a third display example in the case where three or more options are present.

FIG. 10 is a view illustrating a first use example of presentation information by a presentation function.

FIG. 11 is a view illustrating a second use example of the presentation information by the presentation function.

FIG. 12 is a view illustrating a third use example of the presentation information by the presentation function.

FIG. 13 is a block diagram illustrating a medical information processing apparatus according to a second embodiment.

FIG. 14 is a flowchart illustrating a notification process of the medical information processing apparatus according to the second embodiment.

FIG. 15 is a view illustrating a notification example by a notification function according to the second embodiment.

FIG. 16 is a block diagram illustrating a medical information processing apparatus according to a third embodiment.

FIG. 17 is a view illustrating an example of training data according to the third embodiment.

### DETAILED DESCRIPTION

In general, according to one embodiment, a medical information processing apparatus includes processing circuitry. The processing circuitry predicts a medical treatment effect of each of a plurality of options that are possibly selected as a medical treatment judgment for a medical care target. The processing circuitry computes, based on a prediction result of the medical treatment effect, a first importance degree relating to an effect common to the options and a second importance degree relating to a difference in effect between the options, with respect to each of one or more features that affect the medical treatment effect. The processing circuitry presents the first importance degree and the second importance degree by one graph or one list.

Hereinafter, a medical information processing apparatus, method and program according to embodiments are described with reference to the accompanying drawings. Note that in the embodiments below, parts denoted by identical reference signs are assumed to perform similar operations, and an overlapping description is omitted unless where necessary.

### (First Embodiment)

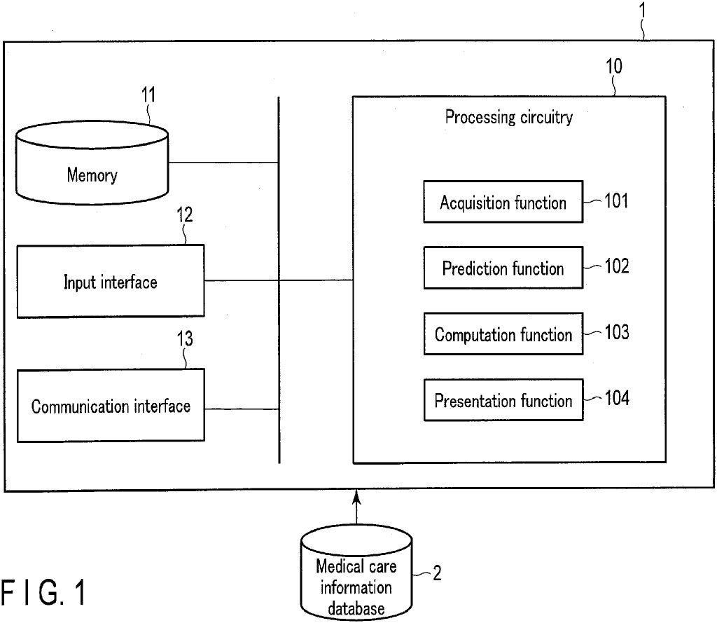

A medical information processing apparatus according to a first embodiment is described with reference to a block diagram of FIG. 1.

A medical information processing apparatus 1 according to the first embodiment includes processing circuitry 10, a memory 11, an input interface 12 and a communication interface 13.

Note that the medical information processing apparatus 1 according to the embodiment described in the present specification may be included in a console, a workstation or the like, or may be included in a medical image diagnosis apparatus such as an MRI (Magnetic Resonance Imaging) apparatus or a CT (Computed Tomography) apparatus.

The processing circuitry 10 includes an acquisition function 101, a prediction function 102, a computation function 103 and a presentation function 104. The processing circuitry 10 includes a processor (not illustrated) as a hardware resource.

The acquisition function 101 acquires patient information, for example, from a medical care information database 2.

The prediction function 102 predicts a medical treatment effect of each of a plurality of options, which may possibly be selected as medical treatment judgments for a medical care target.

The computation function 103 computes, based on the prediction result of the medical treatment effect, a first importance degree relating to an effect common to options (hereinafter referred to as “baseline effect”) with respect to each of one or more features that affect the medical treatment effect. In addition, the computation function 103 computes a second importance degree relating to a difference in effect between the options, with respect to each of one or more features.

The presentation function 104 presents to users the first importance degree and the second importance degree by one graph or one list, for example, on a display (not illustrated). Here, the users include a medical worker represented by a doctor, and a patient.

Note that the various functions in the processing circuitry 10 may be stored in the memory 11 in the form of computer-executable programs. In this case, it can also be said that the processing circuitry 10 is a processor that reads programs corresponding to the various functions from the memory 11 and executes the programs, thereby implementing the functions corresponding to the programs. In other words, the processing circuitry 10 in a state in which the programs are read includes the functions or the like illustrated in FIG. 1 within the processing circuitry 10.

Note that in FIG. 1, the various functions are described as being implemented by the single processing circuitry 10, but a plurality of independent processors may be combined to construct the processing circuitry 10, and the respective processors may execute programs to implement the functions. In other words, there may be a case where the above-described functions are constituted as programs and one processing circuitry executes the programs, or there may be a case where a specific function is implemented in dedicated independent program execution circuitry.

The memory 11 stores various data to be described later, a trained model, and the like. The memory 11 is a semiconductor memory element such as a RAM (Random Access Memory) or a flash memory, a hard disk drive (HDD), a solid state drive (SSD), an optical disc, or the like. Besides, the memory 11 may be a CD-ROM drive, a DVD drive, or a drive unit that reads and writes various information from and to a portable storage medium such as a flash memory.

The input interface 12 includes circuitry that receives various instructions and information inputs from the user. The input interface 12 includes, for example, circuitry relating to a pointing device such as a mouse, or an input device such as a keyboard. Note that the circuitry included in the input interface 12 is not limited to circuitry relating to physical operational components such as a mouse and a keyboard. For example, the input interface 12 may include electric signal processing circuitry that receives an electric signal corresponding to an input operation from an external input device provided separately from the medical information processing apparatus 1, and outputs the received electric signal to various circuitry in the medical information processing apparatus 1.

The communication interface 13 executes exchange of data with an external apparatus by wire or wirelessly. As regards the communication method and the structure of the interface, since general communication means may be used, a description thereof is omitted here.

In addition, the medical information processing apparatus 1 is communicably connected to the medical care information database 2, for example, via a network (not illustrated) and the communication interface 13. Note that the medical information processing apparatus 1 and the medical care information database 2 may be directly connected.

The medical care information database 2 stores patient information relating to one or more patients. The patient information includes information relating to a patient, for example, the age of the patient, the gender, the area of living, and the medical history such as diabetes.

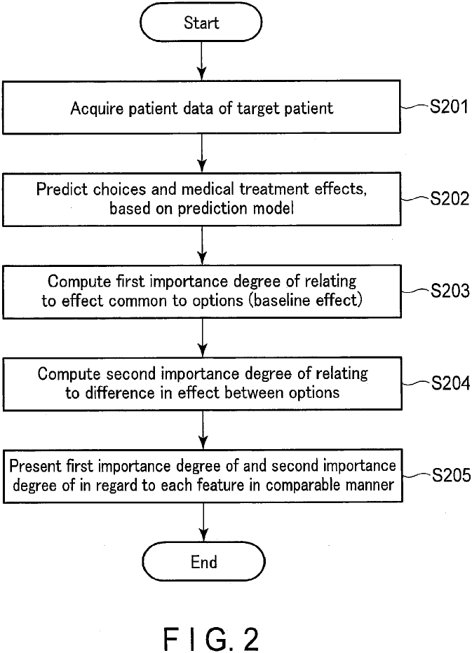

Next, referring to a flowchart of FIG. 2, a description is given of an operation example of the medical information processing apparatus 1 according to the first embodiment.

In step S201, the acquisition function 101 acquires patient information of a target patient as a medical care target.

In step S202, the prediction function 102 predicts, based on the patient information and a prediction model generated in advance, a plurality of options that may possibly be selected as medical treatment judgments, and medical treatment effects of the respective options. Examples of the options include surgery, medication, and follow-up. Examples of the medical treatment effects include a period until recovery, and a survival duration (prognosis period). The prediction model is a model to which the patient information is input, and which outputs a plurality of options and medical treatment effects of the respective options, and, as the prediction model, a prediction model trained by machine learning is assumed. Concretely, a trained prediction model generated in a third embodiment (to be described later) is assumed, but the prediction model is not limited to this and may be any model to which the patient information is input and which outputs options and medical treatment effects of the respective options.

In step S203, based on a prediction result of a medical treatment effect by the prediction unit, the computation function 103 computes a first importance degree relating to a baseline effect, with respect to one or more features. The feature is a parameter that may become a factor affecting (contributing to) the prediction result relating to the option, and examples of the feature include the age of a patient, the gender, the stage, the medical history, and the area of living.

In step S204, the computation function 103 computes a second importance degree relating to a difference in effect between options, in regard to one or more features.

In step S205, the presentation function 104 presents the first importance degree and the second importance degree by one graph or one list in a comparable manner on a feature-by-feature basis. For example, the presentation function 104 displays the first importance degree and the second importance degree by one graph as a cumulative bar graph, on a feature-by-feature basis. Alternatively, the presentation function 104 may display the features by one distribution graph on two-dimensional coordinates with a first axis indicating the first importance degree and a second axis indicating the second importance degree. Alternatively, the presentation function 104 may display the value of the first importance degree and the value of the second importance degree in a list form in regard to each feature. Note that a graph and a list may be combined and displayed.

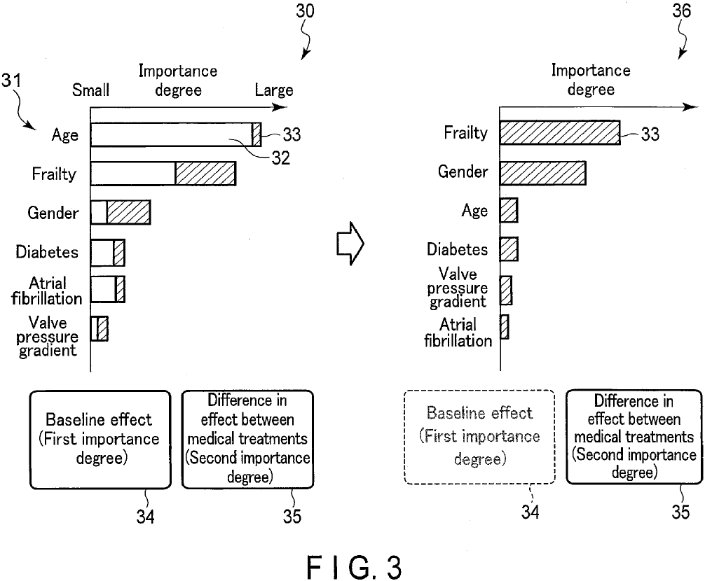

Next, referring to FIG. 3, a description is given of a first presentation example of the first importance degree and the second importance degree by the presentation function 104.

A graph 30 illustrated in a left part of FIG. 3 displays, as a cumulative bar graph, a bar graph of a first importance degree 32 and a second importance degree 33 with respect to each of features 31. Specifically, as the features 31, “Age”, “Frailty”, “Gender”, “Diabetes”, “Atrial fibrillation”, and “Valve pressure gradient” are exemplarily illustrated. With respect to each feature 31, the second importance degree 33 is displayed by being stacked after the bar graph of the first importance degree 32. Note that in the example of FIG. 3, the values of the first importance degree 32 and second importance degree 33 are displayed by normalized values, but the display mode is not limited to this. In addition, an icon 34 of a baseline effect for representing a state in which the first importance degree is displayed, and an icon 35 relating to a difference in effect between options for representing a state in which the second importance degree is displayed, and relating to a difference between medical treatments in the description below, are displayed under the graph 30. Note that the icons 34 and 35 and the graph 30 may be displayed in any positional relationship. In the graph 30, the features 31 may be displayed in the order of the magnitude of the added value of the first importance degree and the second importance degree. In the graph 30 in the left part of FIG. 3, the added value of the first importance degree and the second importance degree is highest in regard to the feature 31 “Age”, and is second highest in regard to the feature 31 “Frailty”.

A graph 36 illustrated in a right part of FIG. 3 illustrates a display example at a time when the icon 35 of the difference between medical treatments has been selected in the display state of the graph 30 of the left part of FIG. 3. For example, by selecting the icon 35 of the difference between medical treatments, only the bar graph of the second importance degree 33 is displayed. Here, in order to improve the visibility, the state is assumed in which the values of only the second importance degrees 33 are normalized, but the magnitudes of the bar graphs in the left part of FIG. 3 may be displayed as such. The contour of the selected icon 35 of the difference between medical treatments is displayed with emphasis by a thick line, and the contour of the unselected icon 34 of the baseline effect is displayed by a broken line. Note that any display mode is possible if the selected importance degree can be distinguished, such as by displaying the unselected icon faintly or in gray, while keeping the selected icon unchanged.

In addition, by a user instruction, either the first importance degree or the second importance degree may be displayed singly. Alternatively, like so-called toggle display, the first importance degree or the second importance degree may be automatically switched at predetermined intervals and may be displayed singly.

In the graph 36, since only the second importance degrees 33 are selected and displayed, the second importance degree 33 of the feature 31 “Frailty” is highest, and the second importance degree 33 of the feature 31 “Gender” is second highest. Thus, the order of display of the features 31 is switched to the descending order of the values of the features 31.

In this manner, by switching the graph display according to the importance degree to which attention is paid, the user, such as a doctor, can easily understand the importance degrees relating to the features affecting the baseline effect, the features affecting the difference between medical treatments, and the features affecting both the baseline effect and the difference between medical treatments.

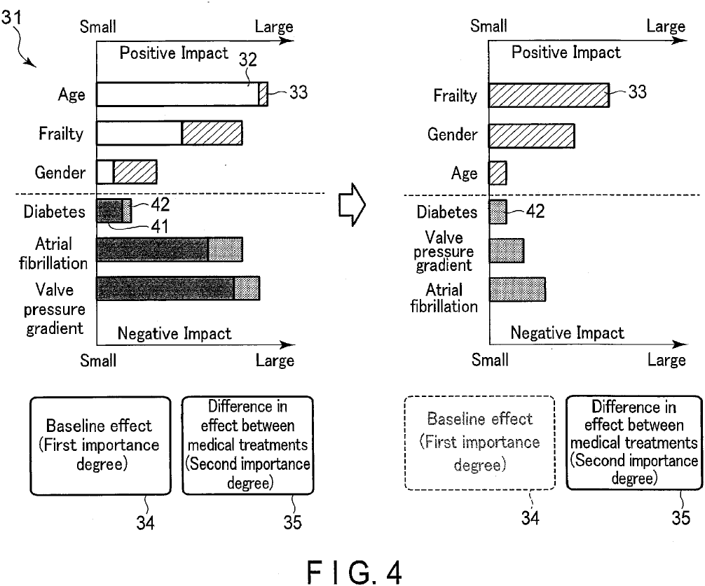

Next, referring to FIG. 4, a description is given of a second presentation example of the first importance degree and the second importance degree by the presentation function 104.

Like FIG. 3, FIG. 4 illustrates an example in which the first importance degree 32 and the second importance degree 33 in regard to each feature 31 are displayed as a cumulative bar graph. However, FIG. 4 differs from FIG. 3 in that in FIG. 3 each of the first importance degree 32 and the second importance degree 33 is displayed as a importance degree of each feature 31 that positively affects (contributes to) the baseline effect or the difference between medical treatments (hereinafter referred to as “positive impact”), whereas in FIG. 4 a importance degree that negatively affects (contributes to) the baseline effect or the difference between medical treatments (hereinafter “negative impact”) is displayed in addition to the positive impact.

For example, as illustrated in a left part of FIG. 4, the features 31 “Age”, “Frailty” and “Gender” are positive impacts, and each of the first importance degree 32 and the second importance degree 33 is displayed as a cumulative bar graph. On the other hand, the features 31 “Diabetes”, “Atrial fibrillation” and “Valve pressure gradient” are negative impacts, and each of a first importance degree 41 and a second importance degree 42 is displayed as a cumulative bar graph.

A right part of FIG. 4 is similar to the right part of FIG. 3, and is a graph in a case where the icon 35 of the difference between medical treatments is selected, i.e., in a case where only the second importance degrees are displayed. As illustrated in the right part of FIG. 4, only the second importance degrees 33 are displayed in regard to the positive impact, and only the second importance degrees 42 are displayed in regard to the negative impact. Thus, like the case of FIG. 3, the importance degrees relating to the baseline effect and the difference between medical treatments can be understood at a glance in regard to each of the features.

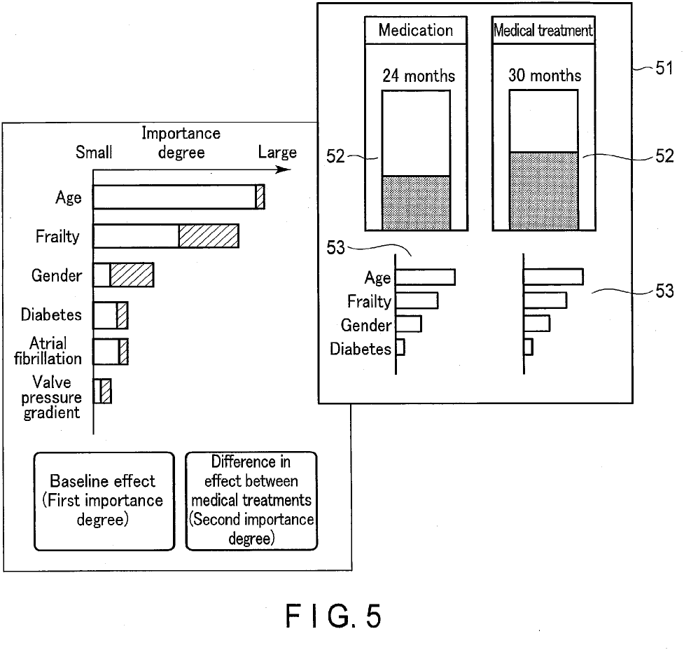

Next, referring to FIG. 5, a description is given of a third presentation example of the first importance degree and the second importance degree by the presentation function 104.

FIG. 5 displays the importance degree for each of features in regard to each of options. For example, by executing an action such as clicking, touching or mousing over the graph illustrated in FIG. 3 or FIG. 4, a graph 51 indicating the importance degree for each feature in regard to each option may be generated by the presentation function 104, and a different window including the graph 51 may be displayed. In the case of displaying the different window, from the standpoint of comparison, it is assumed that the different window is displayed in such a manner as not to overlap the cumulative bar graph like the graph 30, and may be displayed in a superimposed manner.

In the graph 51, “medication” and “medical treatment” are present as options here, and graphs 52 indicating the survival durations and graphs 53 of the importance degrees of the features affecting each option are displayed in regard to each of the “medication” and “medical treatment”. As the graphs 52 and graphs 53, use may be made of prediction results for each option, which are inferred by the prediction function 102 by using a prediction model.

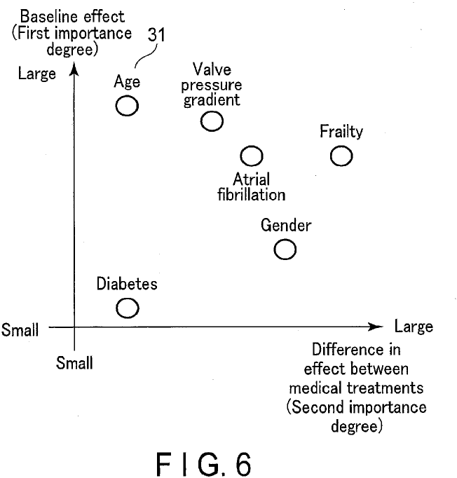

Next, referring to FIG. 6, a description is given of a fourth presentation example of the first importance degree and the second importance degree by the presentation function 104.

FIG. 6 is a distribution graph of the features 31 on a two-dimensional plane with a first axis indicative of the baseline effect and a second axis indicative of the difference between medical treatments. For example, as regards the feature 31 “Age”, it is understood that the first importance degree relating to the baseline effect is high, while the second importance degree relating to the difference between medical treatments is low. In addition, as regards the feature 31 “Diabetes”, it is understood that both the first importance degree relating to the baseline effect and the second importance degree relating to the difference between medical treatments are low. Note that, aside from the cumulative bar graphs and distribution graph illustrated in FIG. 3 to FIG. 6, the first importance degrees and the second importance degrees of the features may be displayed by one graph in any form, if the first importance degrees and the second importance degrees are displayed in a comparable form.

In the above-described examples, as regards the second importance degree relating to the difference between medical treatments, the difference between two options is assumed, but there is a case where three or more options are present. In this case, the second importance degrees may be computed in regard to each of combinations of two, and the computed second importance degrees may be aggregated.

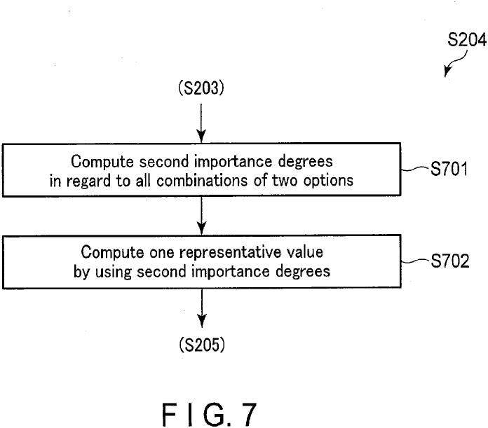

Referring to a flowchart of FIG. 7, a description is given of the details of the process of step S204 by the computation function 103 in a case where three or more options are present.

In step S701, the computation function 103 computes the second importance degrees in regard to all combinations of two options. For example, if aortic valvular stenosis is taken as an example, three options, i.e, surgical aortic valve replacement (SAVR), transcatheter aortic valve implantation (TAVI), and medication are assumed. In this case, the second importance degrees are computed in regard to ₃C₂ = 3 combinations, i.e., three combinations of “SAVR-TAVI”, “SAVR-medication” and “medication-TAVI”. In other words, if there are an n-number of options (n = a natural number of 3 or more), ₙC₂ importance degrees are computed.

In step S702, the computation function 103 computes one representative value by using a plurality of second importance degrees computed in step S701. The computation method of the representative value may be implemented by a statistical process of a maximum value, an average value, a median or the like of the second importance degrees.

Besides, the computation function 103 may compute information of dispersion, such as a standard deviation or a variance, and the information of dispersion may be presented along with the representative value. Note that the computation method of the first importance degree is similar between the case of two options and case of three or more options. In the subsequent steps, the representative value may be used as the second importance degree in step S205.

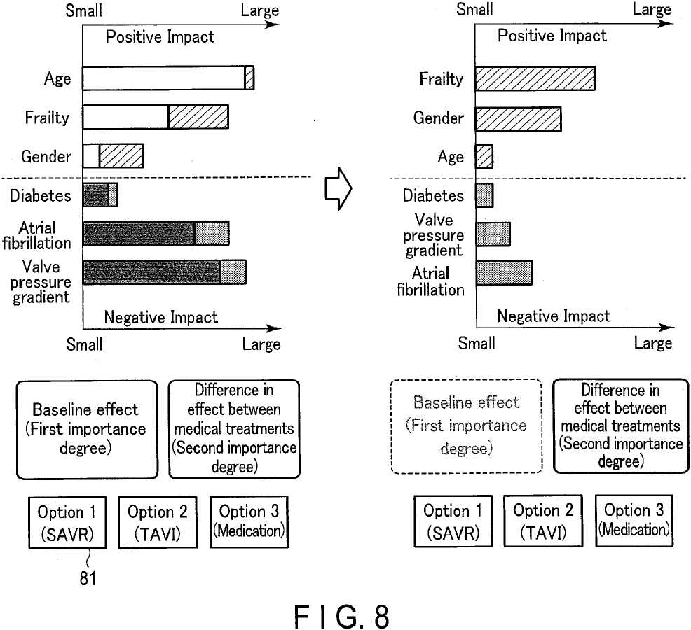

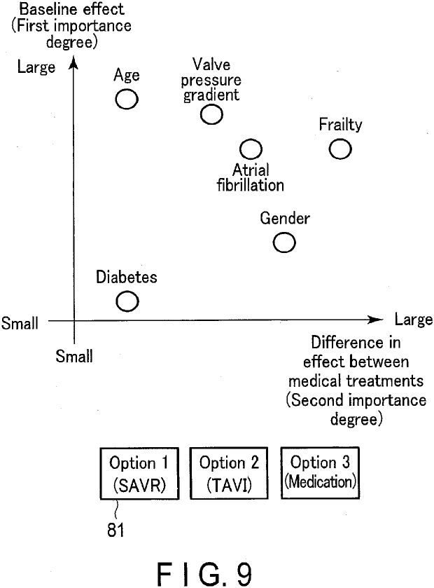

Next, referring to FIG. 8 and FIG. 9, a description is given of a presentation example of the presentation unit in a case where three or more options are present.

FIG. 8 illustrates a display example similar to the cumulative bar graph illustrated in FIG. 4, and FIG. 9 illustrates a display example similar to the distribution graph illustrated in FIG. 6, in the case where three or more options are present. Here, information relating to a plurality of options is displayed by icons 81. In the example of FIG. 8, the information relating to the options is displayed under the icons of the baseline effect (first importance degree) and the difference between medical treatments (second importance degree), and in the example of FIG. 9, the information is displayed under the graph. Note that the icons 81 may be displayed at any position on the screen, if there is no influence on the visual recognition of the graph. In this manner, also in the case of three or more options, like the case of two options, the difference between medical treatments can be displayed.

Note that, aside from displaying the representative value as the difference between medical treatments, two options among the icons of the options may be selected, and the difference between medical treatments relating to the selected two options may be displayed. For example, in FIG. 8, in a case where two options, i.e., option 1 (SAVR) and option 2 (TAVI), are selected, the second importance degree computed between the option 1 (SAVR) and the option 2 (TAVI) may be displayed in place of the average of three second importance degrees.

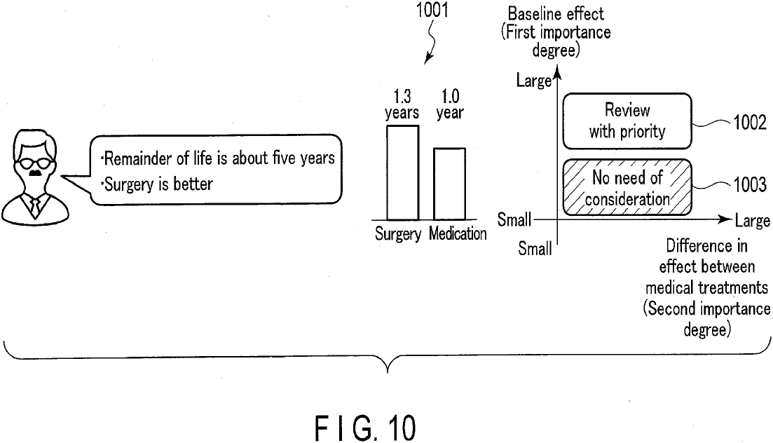

Next, referring to FIG. 10, a description is given of a first use example of the presentation information by the presentation function 104.

In FIG. 10, a scene is assumed in which a doctor that is a user examines a patient, and decides on a survival duration (prognosis period) and a medical treatment policy. Here, it is assumed that the doctor predicts “Remainder of life is about five years” and “Surgery is better” in regard to the patient. On the other hand, the medical information processing apparatus 1 according to the first embodiment computes options of the medical treatment policy and the effect of each option, i.e., a survival duration 1001, in regard to the patient, and the result is that the survival duration is “1.3 years” in the case of option “Surgery”, and the survival duration is “1.0 year” in the case of option “Medication”.

That the survival duration is longer in the option “Surgery” is the same between the doctor’s prediction and the processing result of the medical information processing apparatus 1, but the survival duration that is the processing result of the medical information processing apparatus 1 is about one year and is much shorter than five years that the doctor predicts. In this case, the doctor should grasp which feature affects the survival duration, in regard to the processing result of the medical information processing apparatus 1. Thus, in the example of FIG. 10, it is considered that in the distribution graph presented by the presentation function, a region 1002, in which features greatly affecting the baseline, i.e., features with high first importance degrees, are distributed, is reviewed with priority, and a region 1003, in which features less affecting the baseline, i.e., features with low first importance degrees, are distributed, requires no consideration. In other words, by intensively reviewing the region 1002 in which the features with the high first importance degrees are distributed, the doctor can quickly specify the feature contributing to the inference result that the survival duration is 1.3 years in the case of surgery and is one year in the case of medication.

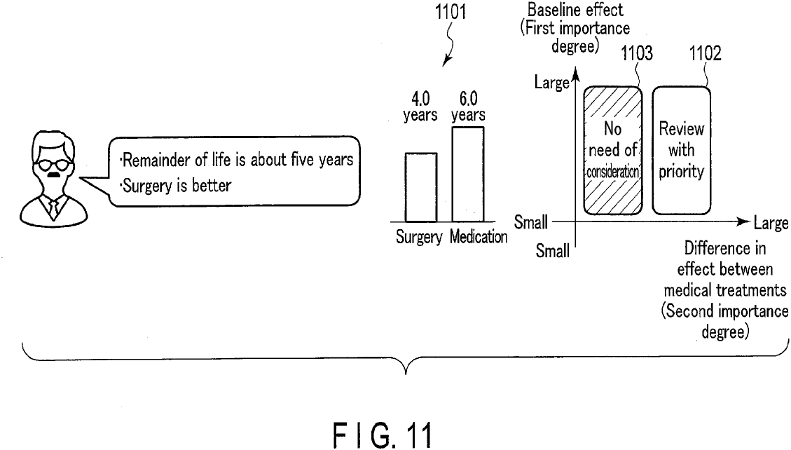

Next, referring to FIG. 11, a description is given of a second use example of the presentation information by the presentation function 104.

FIG. 11 illustrates a case where the doctor predicts similarly with the case of FIG. 10, and, in regard to a survival duration 1101 that is the processing result of the medical information processing apparatus 1, the survival duration is “4.0 years” in the case of the option “Surgery” and the survival duration is “6.0 years” in the case of the option “Medication”.

The survival durations that are output from the medical information processing apparatus 1 do not greatly deviate from the doctor’s prediction, but it is understood that a difference of two years is present between the survival duration in the case of “Surgery” and the survival duration in the case of “Medication”. In this case, the doctor should grasp which feature affects the difference in the survival duration between the options, in regard to the processing result of the medical information processing apparatus 1. Thus, in the example of FIG. 11, it is considered that in the distribution graph presented by the presentation function, a region 1102, in which features greatly affecting the difference between medical treatments, i.e., features with high second importance degrees, are distributed, is reviewed with priority, and a region 1103, in which features less affecting the difference between medical treatments, i.e., features with low second importance degrees, are distributed, requires no consideration. In other words, by intensively reviewing the region 1102 in which the features with the high second importance degrees are distributed, the doctor can quickly specify the feature contributing to the inference result that the difference of two years is present between the survival durations.

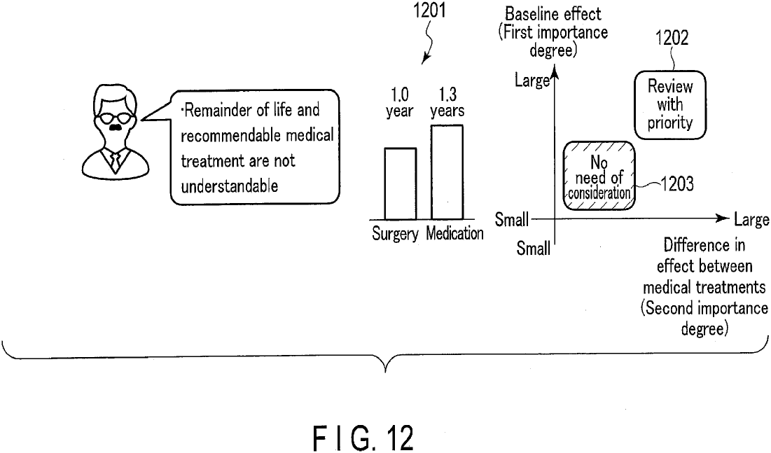

Next, referring to FIG. 12, a description is given of a third use example of the presentation information by the presentation function 104.

In FIG. 12, a case is assumed in which the doctor thinks that “Remainder of life and recommendable medical treatment are not understandable”. At this time, a case is illustrated in which, in regard to a survival duration 1201 that is the processing result of the medical information processing apparatus 1, the survival duration is “1.0 year” in the case of the option “Surgery” and the survival duration is “1.3 years” in the case of the option “Medication”.

In this case, the doctor should grasp which feature affects the survival duration and which feature affects the difference in the survival duration between the options, in regard to the processing result of the medical information processing apparatus 1. Thus, in the example of FIG. 12, it is considered that in the distribution graph presented by the presentation function, a region 1202, in which features greatly affecting both the baseline and the difference between medical treatments, i.e., features with high first importance degrees and high second importance degrees, are distributed, is reviewed with priority, and a region 1203, in which features less affecting the base line and the difference between medical treatments, i.e., features with low first importance degrees and low second importance degrees, are distributed, requires no consideration. In other words, by intensively reviewing the region 1202, the doctor can quickly specify the feature that contributes as the basis of the output of the recommended medical treatment and the survival duration of the recommended medical treatment, i.e., “Medication” and the survival duration “1.3 years” thereof in FIG. 12.

According to the above-described first embodiment, based on the prediction model, the first importance degree, which relates to the baseline effect that is common to a plurality of options, and the second importance degree relating to the difference in effect between the options, are computed, and the first importance degree and the second importance degree are comparably displayed by one graph or one list. For example, the first importance degree and the second importance degree are displayed as a cumulative bar graph in an identical graph. Thereby, the basis of the prediction model can be presented to the user, and, in particular, the user is enabled to easily understand and grasp the feature that greatly affects the medical treatment judgment.

Specifically, in the recommendation-type CDS that proposes options of medical treatment or the like, since the result is interpretable, the patient and the doctor can interpret the result with consent. In addition, since the importance degrees of the features and the effect in regard to the result of CDS can be grasped, the doctor can easily compare and adjust the result of the CDS and the basis of the doctor’s own judgment. In other words, a useful prediction basis can be presented in medical care support.

### (Second Embodiment)

In a second embodiment, it is assumed that advance information relating to features and a user’s judgment criterion is acquired.

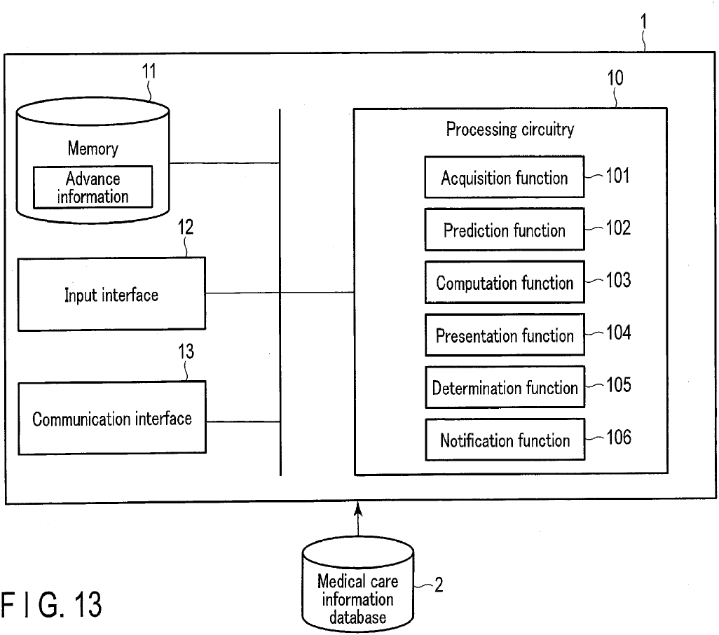

A medical information processing apparatus according to the second embodiment is described with reference to a block diagram of FIG. 13. A medical information processing apparatus 1 according to the second embodiment differs from the medical information processing apparatus 1 according to the first embodiment, in that the processing circuitry 10 further includes a determination function 105 and a notification function 106, in addition to the configuration of the medical information processing apparatus 1 according to the first embodiment.

The memory 11 stores advance information relating to the features and the user’s judgment criterion. Examples of the advance information of the features include a reliability and a measurement date/time. Concretely, since the feature “Frailty” is a subjective judgment, the feature “Frailty” varies greatly from evaluator to evaluator. Thus, the information that the reliability of the feature “Frailty” is low may be stored as advance information. In addition, as regards the measurement date/time, a condition is stored as to whether a latest measurement date/time is a date/time that is a predetermined period or more before a prediction time point by the medical information processing apparatus 1. Note that the measurement date/time may be incorporated as a condition of the reliability. In other words, in addition to a condition that the feature itself is low in reliability, such a condition may be set that a feature measured a predetermined period or more before is low in reliability. In addition, an example of the advance information relating to the user’s judgment criterion is a point on which importance is placed in judgment. Concretely, if a doctor that is a user has a judgment propensity that the doctor places importance on the baseline effect, this judgment propensity may be stored as advance information. Note that the advance information may not necessarily be stored in the memory 11, but may be stored in an external database such as the medical care information database 2, and the advance information may be referred to when the prediction process of the medical information processing apparatus 1 is executed.

The determination function 105 compares the advance information relating to the features, and the features that are output from the prediction model, and determines whether there is a feature that agrees with the advance information. For example, if the feature “Frailty” is present in both the advance information and the features output from the prediction model, it can be said that there is a feature that agrees with the advance information. In addition, if the measurement date/time of the feature “Valve pressure gradient” is a date/time that is a predetermined period or more before the prediction time point by the medical information processing apparatus 1, it is determined that a feature agreeing with the advance information is included. In addition, the determination function 105 determines whether the judgment criterion of a user using the medical information processing apparatus 1 is included in the advance information relating to judgment. For example, in a case where there is advance information that a certain user A places importance on the baseline effect, and if the user A utilizes the medical information processing apparatus 1, the determination function 105 determines that the user’s judgment criterion is included in the advance information.

The notification function 106 notifies the user of the features that are to be reviewed with priority, based on the determination result by the determination function 105 and the first importance degree and second importance degree computed by the computation function 103.

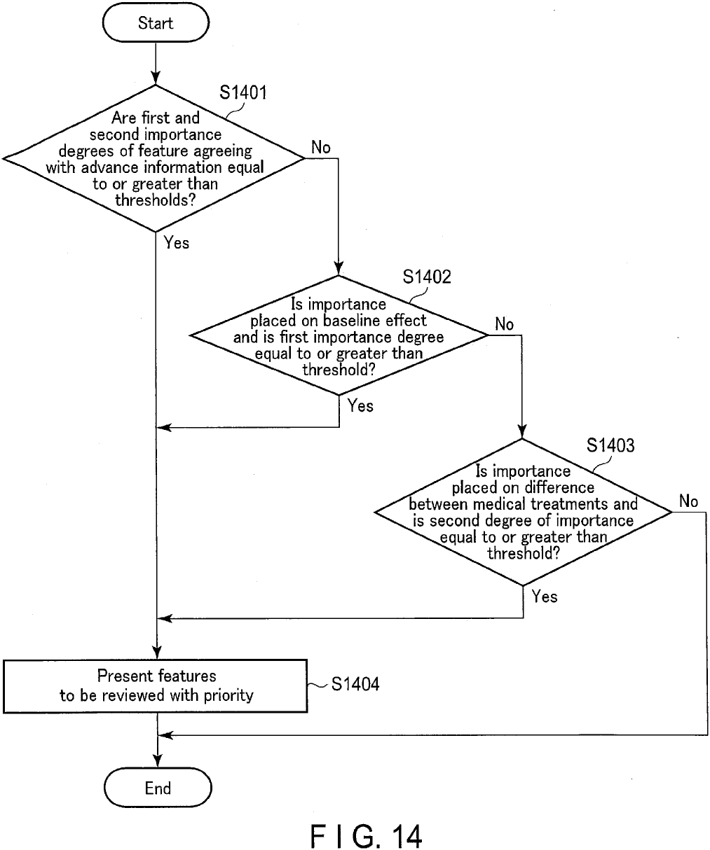

Next, referring to a flowchart of FIG. 14, a description is given of a notification process of the medical information processing apparatus 1 according to the second embodiment.

In step S1401, based on the advance information and the first importance degree and the second importance degree relating to each feature by the computation function 103, the determination function 105 determines whether a feature agreeing with the advance information is present and the first importance degree and second importance degree of this feature are thresholds or more. If a feature agreeing with the advance information is present and the first importance degree and second importance degree of this feature are equal to or greater than the thresholds, the process advances to step S1404, and if the condition is not satisfied, the process advances to step S1402.

In step S1402, the determination function 105 determines whether the point, on which the user places importance in judgment, is the baseline effect and the first importance degree of the feature is equal to or greater than a threshold. If the point, on which the user places importance in judgment, is the baseline effect and the first importance degree of the feature is equal to or greater than the threshold, the process advances to step S1404, and if the condition is not satisfied, the process advances to step S1403.

In step S1403, the determination function 105 determines whether the point, on which the user places importance in judgment, is the difference between medical treatments and the second importance degree of the feature is equal to or greater than a threshold. If the point, on which the user places importance in judgment, is the difference between medical treatments and the second importance degree of the feature is equal to or greater than the threshold, the process advances to step S1404, and if the condition is not satisfied, the process ends.

In step S1404, the notification function 106 notifies the relevant feature is the feature that is to be reviewed with priority at a time of judgment by the user. For example, if the feature “Frailty” is included as the advance information, and “Frailty” is present as the feature of the prediction model, and the first importance degree and the second importance degree are thresholds or more, the user can be notified that the “Frailty” should be intensively reviewed since the importance degree of “Frailty” is high in the prediction result, although the “Frailty” is a feature with low reliability.

In addition, if there is advance information that the user places importance on the difference in effect between the options, for example, if the prediction by the user is the surgery and the prediction result from the medical information processing apparatus 1 is the medication, it is considered that the present situation is a situation in which the user places importance on the difference in effect between the options. Here, if the second importance degree of a certain feature is a threshold or more, the user can be notified that this feature should be reviewed with priority.

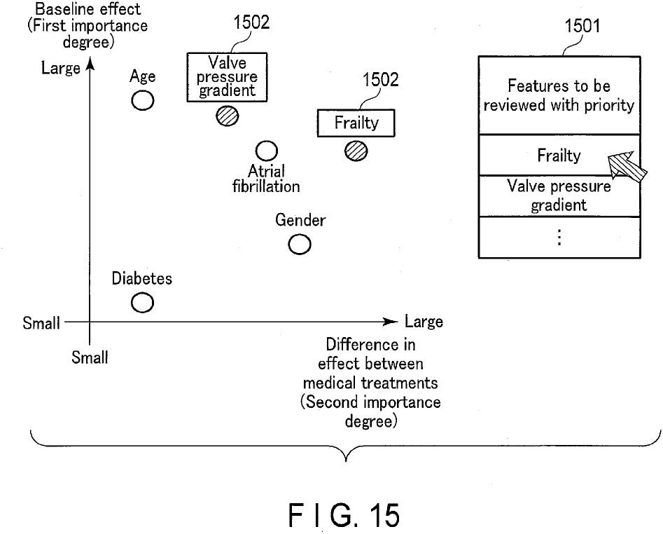

Next, referring to FIG. 15, a description is given of a notification example by the notification function 106 according to the second embodiment.

FIG. 15 illustrates an example in which features to be reviewed with priority are displayed by a list 1501 by the notification 106 and the presentation function 104. Although it is assumed here that the list 1501 is displayed along with a distribution graph as illustrated in FIG. 6, the list 1501 may be displayed independently from the distribution graph. The notification function 106 may set the degree of priority in the order from the highest first importance degree or second importance degree, and the presentation function 104 may display features 1502 to be reviewed in the list 1501 in the order of priority. The presentation function 104 may execute, together with the list display or in place of the list display, the display with emphasis, such as by flickering plots representing the features 1052 to be reviewed with priority in the distribution graph, or by displaying character strings immediately above the plots by boxing them or by boldface, or by making the color of the plots of the features 1052 different from the color of the plots of the other features. In other words, any display mode may be adopted if the features 1052 to be reviewed with priority can be distinguished from the other features.

In addition, in a case where a feature displayed on the list 1501 is selected by a cursor or the like, the presentation function 104 may display only the selected feature 1502 with emphasis on the distribution graph. Moreover, detailed information of the selected feature 1502 may be displayed by a pop-up or the like. Concretely, for example, if the cursor is superimposed (so-called “mouse-over”) on any one of a character string of a feature displayed on the list 1501, a plot of the feature, and a character string above the plot, detailed information such as an actual measured value relating to the feature, a measurement value and raw data that is the basis are displayed by pop-up. Thereby, the first importance degree and second importance degree of the feature, which the user wishes to confirm, become easier to understand, and the detailed information can quickly be confirmed.

Note that since it can be said that a feature with high priority is important in medical treatment judgment, it is possible to recommend the order of features to be acquired, such as in a case of examining the features in the order from the feature with the highest priority. Conversely, the notification function 106 may notify a feature, the measurement of which can be omitted.

For example, before acquiring the patient data of a target patient, the prediction function 102 allocates, for example, a random number to a feature that a target of processing (hereinafter “target feature”), and computes a prediction result by using a prediction model along with other features. The computation function 103 computes the first importance degree and second importance degree from the prediction result. By the determination function 105, if the computed first importance degree and second importance degree are thresholds or less, it can be determined that the feature is unnecessary in medical treatment judgment, regardless of the actual value or random number. For example, if the feature “blood-cholesterol level” is determined to be an unnecessary feature, a blood examination can be omitted, and therefore a medical treatment judgment with an equal precision to the case of performing all examinations can be supported with a less number of examinations, leading to reduction in cost and time.

According to the above-described second embodiment, by the notification function, additional support information, such as a notification of features that are to be intensively reviewed according to the importance degrees of features, is presented. Thereby, since important features, among many features, can be narrowed down and reviewed, the efficiency of the user’s judgment is improved. In addition, by omitting the measurement of features of less importance degrees, a medical treatment judgment with equal precision to the case of performing complete examinations can be supported with a less number of examinations.

### (Third Embodiment)

In a third embodiment, a medical information processing apparatus 1 includes a training function, and generates a prediction model by training a model by using training data.

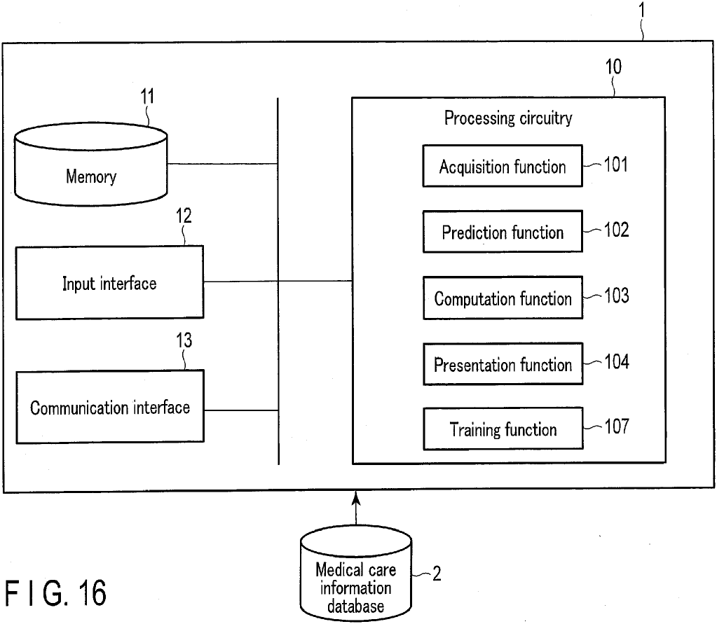

The medical information processing apparatus 1 according to the third embodiment is described with reference to a block diagram of FIG. 16.

The medical information processing apparatus 1 illustrated in FIG. 16 includes a training function 107, in addition to the processing circuitry 10 according to the first embodiment.

The memory 11 according to the second embodiment may store training data and a pre-training model. Alternatively, each time a training process by the medical information processing apparatus 1 is executed, training data and a pre-training model may be acquired from the medical care information database 2 and may be stored in the memory 11. The training data is data relating to past medical care judgments and medical treatment results, and is data that is a set of values relating to one or more features in regard to a plurality of medical care targets in the past, i.e., a plurality of patients in the past, options selected for the patients in the past, and medical treatment results by the selected options.

The training function 107 generates a prediction model by training a model by using training data. Specifically, the training function 107 generates a prediction model to which values relating to one or more features are input, and which outputs medical treatment effects of individual options. In addition, based on the prediction model, the training function 107 generates a first importance degree prediction model that outputs a first importance degree, and a second importance degree prediction model that outputs a second importance degree. Furthermore, in a case where the first importance degree and second importance degree of a feature are difficult to obtain by a prediction model alone, such as where the prediction model is a nonlinear model, the training function 107 generates, by training, an explanatory model for explaining the importance degree of the feature. Needless to say, the explanatory model may be generated even in the case where the prediction model is a linear model.

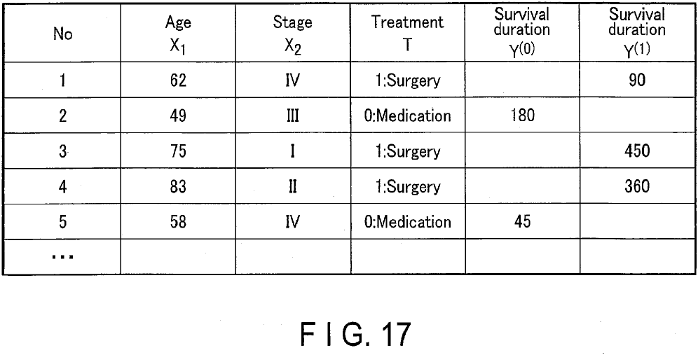

Next, an example of the training data according to the third embodiment is described with reference to FIG. 17.

FIG. 17 is a table illustrating patient information based on past results of medical treatments. Here, a number serving as an ID, an age X₁, a stage X₂ indicative of the degree of progress of a disease, a treatment T, and survival durations Y⁽⁰⁾ and Y⁽¹⁾ that are outcomes, are correlated. The age and the stage are examples of features, and two features are illustrated here, but, aside from this, an i-number of features (i is a natural number of 2 or more) may be present. The treatment T indicates which option was selected as a medical treatment of the patient, and it is assumed here that “0” is set for medication and “1” is set for surgery. The survival duration Y⁽⁰⁾ is a survival duration in a case where the medication was selected as the medical treatment, and the survival duration Y⁽¹⁾ is a survival duration in a case where the surgery was selected as the medical treatment. Note that since only one option is selected as the medical treatment for one patient, or, in other words, since only the medication or the surgery is selected here, the information of only the survival duration Y⁽⁰⁾ or the survival duration Y⁽¹⁾ is present for one patient.

A description is now given of a first training method of a prediction model and a computation model of a importance degree using the training data as illustrated in FIG. 17. As the first training method, a method is described in which T-Learner that is a kind of Meta-Learner, which is a framework that infers an individual causal effect (individual treatment effect (ITE)) by machine learning, is used as a prediction model that predicts a medical treatment effect (here, a survival duration) for each option of medical treatment, and a linear regression model is assumed. Here, for the purpose of convenience of description, two kinds, i.e., medication and surgery, are assumed as options, and two kinds, i.e., X₁ and X₂, are assumed as features, but training can be performed by the same method even in the case of three or more kinds.

To begin with, the training function 107 generates a group for each option from training data. Specifically, the training function 107 classifies patient information into a group of patient information in which medication is selected as medical treatment, and a group of patient information in which surgery is selected as medical treatment.

Next, the training function 107 trains, by supervised learning, a model that predicts an outcome Y for each option by using a feature Xᵢ. Specifically, in the example of FIG. 17, in the case of a model relating to medication as indicated in equation (1) below, coefficients of α₀ and β₀ in equation (1) are trained by training the model such that features X₁ and X₂ are input and a survival duration Y⁽⁰⁾ is output. Note that γ₀ is a bias.

$${\text{Y}}^{\left(0\right)} = {\text{α}}_{0} {\text{X}}_{1} {}^{\left(0\right)} + {\text{β}}_{0} {\text{X}}_{2} {}^{\left(0\right)} + {\text{γ}}_{0}$$

Note that a superscript (0) added to each of the features X₁ and X₂ is indicative of training data of a group of patient information in which medication is selected.

Similarly, in the case of a model relating to surgery as indicated in equation (2) below, coefficients of α₁ and β₁ in equation (2) are trained by training the model such that features X₁ and X₂ are input and a survival duration Y⁽¹⁾ is output. Note that γ₁ is a bias.

$${\text{Y}}^{\left(1\right)} = {\text{α}}_{1} {\text{X}}_{1} {}^{\left(1\right)} + {\text{β}}_{1} {\text{X}}_{2} {}^{\left(1\right)} + {\text{γ}}_{1}$$

Note that a superscript (1) added to each of the features X₁ and X₂ is indicative of training data of a group of patient information in which surgery is selected. Since the above-described training of the prediction model for each option is similar to the training of an estimation model of an outcome in T-Learner, a concrete description thereof is omitted.

Here, in a case where medication is regarded as a standard medical treatment, a computation equation of a baseline effect τ^_{B} is similar to equation (1) that is the prediction model relating to medication, and is expressed by equation (3). A computation equation of a difference in effect between medical treatments, τ^_{ITE}, which is an individual treatment effect, is expressed by equation (4). A superscript caret (^) is indicative of an estimation value.

$$\text{τ} {\hat{}}_{\text{B}} = {\text{α}}_{0} {\text{X}}_{1} + {\text{β}}_{0} {\text{X}}_{2} + {\text{γ}}_{0}$$

$$\text{τ} {\hat{}}_{\text{ITE}} = \left({\text{α}}_{1} - {\text{α}}_{0}\right) {\text{X}}_{1} + \left({\text{β}}_{1} - {\text{β}}_{0}\right) {\text{X}}_{2} + {\text{γ}}_{1} - {\text{γ}}_{0}$$

Here, since equation (3) and equation (4) are expressed by linear regression models, it can be said that equation (3) is a first importance degree prediction model. The coefficient α₀ is indicative of the degree of influence on the baseline effect of the feature X₁, i.e., the first importance degree of the feature X₁. The coefficient β₀ is indicative of the degree of influence on the baseline effect of the feature X₂, i.e., the first importance degree of the feature X₂. Similarly, it can be said that equation (4) is a second importance degree prediction model. The value of the coefficient (α₁-α₀) is indicative of the degree of influence on the difference in effect between medical treatments of the feature X₁, i.e., the second importance degree of the feature X₁. Likewise, the coefficient (β₁-β₀) is indicative of the degree of influence on the difference in effect between medical treatments of the feature X₂, i.e., the second importance degree of the feature X₂.

Thus, using equations (3) and equation (4), the computation function 103 can compute the first importance degree and the second importance degree in regard to each feature.

Next, a description is given of a second training method of a prediction model and a computation model of a importance degree in a case where a standard medical treatment is absent.

In the first training method, the medication treatment, as the standard treatment, is set to be an option of the baseline effect. In the case where a definite standard medical treatment is absent, it is necessary to separately generate a prediction model that predicts the baseline effect.

For example, an average of prediction models of respective options relating to medical treatment, i.e., medication and surgery, may be set as a prediction model of the baseline effect, and can be expressed by equation (5).

$$\text{τ} {\hat{}}_{\text{B}} = \left\{\left({\text{α}}_{0} + {\text{α}}_{1}\right) / 2\right\} * {\text{X}}_{1} + \left\{\left({\text{β}}_{0} + {\text{β}}_{1}\right) / 2\right\} * {\text{X}}_{2} + \left({\text{γ}}_{0} - {\text{γ}}_{1}\right) / 2$$

In other words, equation (5) is a first importance degree prediction model, and the value of “(α₀+α₁)/2” is obtained as the first importance degree of the feature X₁, and the value of “(β₀+β₁)/2” is obtained as the second importance degree of the feature X₂. Note that the difference in effect between medical treatments, τ^_{ITE}, and the second importance degree may be computed by using equation (4).

In another example, instead of generating prediction models for medication and surgery separately, a prediction model may be trained without the insertion of the term of treatment T, as indicated in, for example, equation (6), by using all training data.

$${\text{Y}}^{\left(\text{i}\right)} = {\text{α}}_{\text{B}} {\text{X}}_{1} {}^{\left(\text{i}\right)} + {\text{β}}_{\text{B}} {\text{X}}_{2} {}^{\left(\text{i}\right)} + {\text{γ}}_{\text{B}} , \text{i} ∈ \left(0 , 1\right)$$

Specifically, without distinguishing the options between medication and surgery, the values of the coefficients α_{B} and β_{B} and the bias γ_{B} in equation (6) are learned by using the training data in which the features X₁ and X₂ are input data and the survival duration that is the outcome is correct answer data. After the training is completed, the prediction model of the baseline effect can be expressed by, for example, equation (7).

$$\text{τ} {\hat{}}_{\text{B}} = {\text{α}}_{\text{B}} {\text{X}}_{1} + {\text{β}}_{\text{B}} {\text{X}}_{2} + {\text{γ}}_{\text{B}}$$

According to equation (7), the value of “α_{B}” is obtained as the first importance degree of the feature X₁, and the value of “β_{B}” is obtained as the second importance degree of the feature X₂.

Note that as the prediction model of the baseline effect, use may be made of a prediction model in which the term of treatment T is inserted as indicated in equation (8). λ is a coefficient.

$$\text{τ} {\hat{}}_{\text{B}} = \text{λΤ} \text{+} {\text{α}}_{\text{B}} {\text{X}}_{1} + {\text{β}}_{\text{B}} {\text{X}}_{2} + {\text{γ}}_{\text{B}}$$

In the case as indicated in equation (8), since the term of treatment T is included in the prediction model, it cannot be said, strictly speaking, that this prediction model is a prediction model of the baseline effect. However, since the first importance degree is determined by the coefficient of each feature, there is no problem even if the term of treatment T is included in the prediction model.

In the above-described example, the case is assumed that the training is executed by equally using the respective patient data included in the training data. However, in general, since the features are non-uniform among the options of medical treatments, there is a possibility that a bias occurs in the prediction result by the prediction model. Thus, the non-uniformity of the training data may be adjusted by using a propensity score.

To begin with, for example, by the training function 107, a propensity score model that computes the propensity score for each option is trained. The propensity score model may be computed by using, for example, logistic regression. Note that since the training of the propensity score model and the computation of the propensity score may be executed by using ordinary methods, a concrete description thereof is omitted here.

Thereafter, based on the propensity score for each option, which is computed by using the propensity score model, the training data is reconstructed by the training function 107. For example, as the propensity score of an option is higher, the propensity to select this option is indicated, and as the propensity score of an option is lower, the propensity to not select this option is indicated. Thus, the patient data in which this option is selected may be increased by a reciprocal number of the propensity score of the option. For example, if the propensity score of surgery is “0.1”, ten patient data are generated from one patient data in which surgery is selected, such that the patient data becomes data of 1/0.1 = 10 persons. The patient data that is generated may be generated by values obtained by randomly allocating the value of the feature of one patient data, which is the generation source, within a predetermined range.

Next, a description is given of a third training method of a prediction model, a first importance degree prediction model, and a second importance degree prediction model.

In the first training method and the second training method, the case was assumed in which the model is a linear model. In the third training method, a case is assumed in which the model is a nonlinear model.

For example, a nonlinear model that outputs an outcome µ^₀ relating to medication, and a nonlinear model that outputs an outcome µ^₁ relating to surgery, can be expressed as indicated by equation (9) and equation (10).

$$μ {\hat{}}_{0} = {\text{M}}_{0} \left({\text{Y}}^{\left(0\right)} \sim {\text{X}}^{\left(0\right)}\right)$$

$$μ {\hat{}}_{1} = {\text{M}}_{1} \left({\text{Y}}^{\left(1\right)} \sim {\text{X}}^{\left(1\right)}\right)$$

Here, M₀ () is a nonlinear model using the feature X⁽⁰⁾ and the survival duration Y⁽⁰⁾ of the patient data relating to a patient for which medication is selected, and M₁() is a nonlinear model using the feature X⁽¹⁾ and the survival duration Y⁽¹⁾ of the patient data relating to a patient for which surgery is selected. The nonlinear model may be a nonlinear model used in general machine learning and statistics, such as a deep neural network, a nonlinear support vector machine, or the like. Note that a general training method used for the training data may be used for the training of the nonlinear models indicated by equation (9) and equation (10).

In the case where the medication treatment, as the standard treatment, is regarded as an option of the baseline, a baseline effect τ^_{B} and a difference in effect between medical treatments, τ^_{ITE}, can be expressed as indicated by equation (11) and equation (12).

$$\text{τ} {\hat{}}_{\text{B}} = μ {\hat{}}_{0} \left(\text{X}\right)$$

$$\text{τ} {\hat{}}_{\text{ITE}} = μ {\hat{}}_{1} \left(\text{X}\right) - μ {\hat{}}_{0} \left(\text{X}\right)$$

Here, in linear models, coefficients of the features can be used as the first importance degree and the second importance degree. However, in nonlinear models as indicated by equation (11) and equation (12), since there are no values corresponding to the coefficients of the features, explanatory models for computing the first importance degree and the second importance degree are prepared.

It is assumed that as the explanatory models, a global explanatory model that converts a complex model to an interpretable model, and a local explanatory model that explains a prediction basis for a specific input, are generated, and the first importance degree and the second importance degree may be computed by using at least one of these models. In the global explanatory model, complex models, such as a random forest and a deep neural network, are approximately expressed by models with high readability, such as a single decision tree and a rule-base model. In the global explanatory model, in the case of a decision tree, since elements that are nodes can be regarded as features, the weight coefficients of the nodes can be used as the first importance degrees and the second importance degrees of the features relating to the nonlinear models M₀ and M₁.

On the other hand, as the local explanatory model, use can be made of LIME (local interpretable model-agnostic explanations), SHAP (Shapley Additive exPlanations), Anchors, and the like, which can quantitatively express the contribution of each feature in a prediction mode, which are methods used in regard to the interpretability and explainability of a machine learning model. Note that since the process for outputting an explainability relating to features in the respective methods is a general method, a description thereof is omitted here.

Note that although the example of using X-Learner for computing the difference in effect between medical treatments, τ^_{ITE}, is illustrated here, aside from this, any general method of computing the individual treatment effect, such as X-Learner, R-Learner, DR-Learner, Causal Forest, or GANITE, is applicable.

According to the above-described third embodiment, a prediction model is generated by training, and if the importance degree of the feature cannot be extracted, such as where the prediction mode is a nonlinear model, an explanatory model for computing the importance degree is trained and generated. Thereby, the explanatory model can be utilized as the prediction model relating to the first embodiment. In addition, even if prediction models relating to the baseline effect and the difference in effect between the options are generated as nonlinear models, and the importance degrees of features cannot simply be computed, the importance degrees of features can be computed by the explanatory model.

According to at least one of the above-described embodiments, a useful prediction basis in medical care support can be presented.

Additionally, the respective functions according to the embodiments can also be implemented by installing programs for executing the corresponding processes in a computer such as a workstation, and by developing the programs on the memory. At this time, the programs that can cause the computer to execute the corresponding method can be distributed by being stored in a storage medium such as a magnetic disk (e.g., hard disk), an optical disc (e.g., CD-ROM, DVD, or Blu-ray (trademark) disc), or a semiconductor memory.

Note that the term “processor” used in the above description means, for example, a CPU (Central Processing Unit), a GPU (Graphics Processing Unit), or circuitry such as an application specific integrated circuit (ASIC) or a programmable logic device (e.g., simple programmable logic device (SPLD), a complex programmable logic device (CPLD) or a field programmable gate array (FPGA)). If the processor is, for example, a CPU, the processor implements the functions by reading and executing the program stored in the storage circuitry. On the other hand, if the processor is, for example, an ASIC, the functions are directly incorporated in the circuitry of the processor as logic circuitry, instead of the programs being stored in the storage circuitry. Note that, aside from the case where each of the processors of the embodiments is constructed as single circuitry for each processer, the processors may be constructed as a single processor by combining a plurality of independent circuits and thereby the functions may be implemented. Furthermore, a plurality of structural elements in the drawings may be integrated into a single processor, and the functions thereof may be implemented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
predict a medical treatment effect of each of a plurality of options that are possibly selected as a medical treatment judgment for a medical care target;
compute, based on a prediction result of the medical treatment effect, a first importance degree relating to an effect common to the options and a second importance degree relating to a difference in effect between the options, with respect to each of one or more features that affect the medical treatment effect; and
present the first importance degree and the second importance degree by one graph or one list.

2. The medical information processing apparatus according to claim 1, wherein the processing circuitry displays the first importance degree and the second importance degree as a cumulative bar graph in regard to each of the features.

3. The medical information processing apparatus according to claim 2, wherein the processing circuitry switching, according to a user instruction, such that the first importance degree or the second importance degree is displayed singly.

4. The medical information processing apparatus according to claim 1, wherein the processing circuitry displays the features on two-dimensional coordinates with a first axis indicating the first importance degree and a second axis indicating the second importance degree.

5. The medical information processing apparatus according to claim 1, wherein the processing circuitry is further configured to:
determine whether or not the first importance degree and the second importance degree are thresholds or more in regard to a first feature included in advance information relating to a reliability of the feature and a judgment criterion of a user; and
notify, in a case where the first importance degree and the second importance degree of the first feature are the thresholds or more, that the first feature is a feature that is to be reviewed with priority.

6. The medical information processing apparatus according to claim 1, wherein the processing circuitry is further configured to:
determine whether or not a first feature with the first importance degree of a threshold or more is present, in a case where a user places importance on the effect common to the options; and
notify, in a case where the first feature is present, that the first feature is a feature that is to be reviewed with priority.

7. The medical information processing apparatus according to claim 1, wherein the processing circuitry is further configured to:
determine whether or not a first feature with the second importance degree of a threshold or more is present, in a case where a user places importance on the difference in effect between the options; and
notify, in a case where the first feature is present, that the first feature is a feature that is to be reviewed with priority.

8. The medical information processing apparatus according to claim 1, wherein the processing circuitry is further configured to determine, in a case where a first feature with the first importance degree and the second importance degree that are thresholds or less is present, that the first feature is an unnecessary feature in the medical treatment judgment.

9. The medical information processing apparatus according to claim 1, wherein the processing circuitry is further configured to generate a prediction model to which a value relating to the one or more features is input and which outputs a medical treatment effect of each of the options, by using, as training data, a value relating to the one or more features in regard to a medical care target in a past, an option selected for the medical care target in the past, and a medical treatment result by the selected option.

10. The medical information processing apparatus according to claim 9, wherein the processing circuitry is further configured to generate, based on the prediction model, a first importance degree prediction model that outputs the first importance degree, and a second importance degree prediction model that outputs the second importance degree.

11. The medical information processing apparatus according to claim 10, wherein the processing circuitry generates at least one of a first explanatory model that explains a basis for an inference result of the first importance degree prediction model, and a second explanatory model that explains a basis for an inference result of the second importance degree prediction model.

12. A medical information processing method comprising:
predicting a medical treatment effect of each of a plurality of options that are possibly selected as a medical treatment judgment for a medical care target;
computing, based on a prediction result of the medical treatment effect, a first importance degree relating to an effect common to the options and a second importance degree relating to a difference in effect between the options, with respect to each of one or more features that affect the medical treatment effect; and
presenting the first importance degree and the second importance degree by one graph or one list.
